(19) United States
(12) Patent Application Publication
(10) Pub. No.: US 2023/0293719 A1
(43) Pub. Date: Sep. 21, 2023

(54) PEPTIDE-LIPOSOME COMPLEX FOR MULTIVALENT CROSSLINKING WITH PD-L1 AND COMPOSITION INCLUDING THE SAME

(71) Applicant: KOREA INSTITUTE OF SCIENCE AND TECHNOLOGY, Seoul (KR)

(72) Inventors: Kwangmeyung KIM, Seoul (KR); In-Cheol SUN, Seoul (KR); Hong Yeol YOON, Seoul (KR); Man Kyu SHIM, Seoul (KR); Suah YANG, Seoul (KR)

(73) Assignee: KOREA INSTITUTE OF SCIENCE AND TECHNOLOGY, Seoul (KR)

(21) Appl. No.: 17/859,078

(22) Filed: Jul. 7, 2022

(30) Foreign Application Priority Data
Jan. 28, 2022 (KR) 10-2022-0013360

(51) Int. Cl. A61K 47/69; A61P 35/00; A61K 47/64

(52) U.S. Cl. CPC A61K 47/6915; A61K 47/64; A61P 35/00

(57) ABSTRACT

Disclosed is an optimal peptide-liposome complex capable of multivalent crosslinking with PD-L1 on the cell surface to induce degradation of PD-L1. The peptide-liposome complex effectively blocks PD-L1, an immune checkpoint on the surface of cancer cells, and prevents the recycling of PD-L1 by intracellular metabolism to induce complete degradation of PD-L1 in cancer cells, achieving an increased therapeutic effect on cancer.

### CROSS-REFERENCE TO RELATED APPLICATIONS

This application claims priority under 35 U.S.C. §119 to Korean Patent Application No. 10-2022-0013360 filed on Jan. 28, 2022 in the Korean Intellectual Property Office, the disclosure of which is incorporated herein by reference in its entirety.

### BACKGROUND OF THE INVENTION

### 1. Field of the Invention

The present invention relates to an optimal peptide-liposome complex capable of multivalent crosslinking with PD-L1 on the cell surface to induce degradation of PD-L1 and a use thereof.

### 2. Description of the Related Art

Cancer immunotherapy using immune checkpoint inhibitors has recently led to significant clinical advances for cancer treatment, with many reported cases of complete recovery from cancer. Particularly, a considerable number of drugs with high therapeutic efficacy are currently used in clinical applications as monoclonal antibodies that selectively bind to immune checkpoints, specifically programmed death-ligand 1 (PD-L1), programmed death-receptor (PD-1), and cytotoxic T lymphocyte associated protein 4 (CTLA-4), which are involved in the interaction between cancer cells and T cells.

However, these monoclonal antibodies are very expensive because they require enormous costs for mass production and quality control. Another problem is that the immunogenicity of the antibodies causes immune-related adverse events fatal to organs. According to the results of recent studies, immune checkpoint inhibition using antibodies may cause recycling of immune checkpoints by intracellular metabolism, leading to resistance to anticancer immunotherapy as well as low efficacy of anticancer immunotherapy. Thus, there is an urgent need to develop a therapeutic agent that can effectively inhibit immune checkpoints, prevent the recycling of immune checkpoints, and induce the intracellular degradation of immune checkpoints.

### PRIOR ART DOCUMENTS

### Patent Documents

(Patent Document 1) Korean Patent Publication No. 10-2011-0000036

### SUMMARY OF THE INVENTION

The present invention has been made in an effort to solve the above-described problems and an object of the present invention is to provide a peptide-liposome complex for multivalent crosslinking with PD-L1 that can effectively inhibit the immune checkpoints and induce intracellular degradation of PD-L1, and a use thereof.

One aspect of the present invention provides a peptide-liposome complex composed of a lipid bilayer including (a) a first phospholipid, (b) a second phospholipid containing PEG, (c) cholesterol, and (d) a lipid conjugate consisting of the second phospholipid and a peptide having the amino acid sequence set forth in SEQ ID NO: 1.

The first phospholipid may be selected from the group consisting of phosphatidylcholine (PC), phosphatidic acid (PA), phosphatidylethanolamine (PE), phosphatidylglycerol (PG), phosphatidylserine (PS), phosphatidylinositol (PI), dimyristoylphosphatidylcholine (DMPC), distearoylphosphatidylcholine (DSPC), dioleoylphosphatidylcholine (DOPC), dipalmitoylphosphatidylcholine (DPPC), dimyristoylphosphatidylglycerol (DMPG), distearoylphosphatidylglycerol (DSPG), dioleoylphosphatidylglycerol (DOPG), dipalmitoylphosphatidylglycerol (DPPG), dimyristoylphosphatidylserine (DMPS), distearoylphosphatidylserine (DSPS), dioleoylphosphatidylserine (DOPS), dipalmitoylphosphatidylserine (DPPS), dioleoylphosphatidylethanolamine (DOPE), palmitoyloleoylphosphatidylcholine (POPC), palmitoyloleoylphosphatidylethanolamine (POPE), dioleoylphosphatidylethanolamine 4-(N-maleimidomethyl)-cyclohexane-1-carboxylate (DOPE-mal), dipalmitoylphosphatidylethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidylethanolamine (SOPE), 1,2-dielaidoyl-sn-glycero-3-phosphoethanolamine (transDOPE), cardiolipin, and mixtures thereof.

The second phospholipid may be 1,2-distearoyl-sn-glycero-3-phosphoethanolamine-N-[methoxy(polyethylene glycol)2000] (DSPE-mPEG2000) or 1,2-disteroyl-sn-glycero-3-phosphoethanolamine-N-[maleimide(polyethylene glycol)2000] (DSPE-PEG2000-MAL).

The lipid conjugate consisting of the second phospholipid and a peptide having the amino acid sequence set forth in SEQ ID NO: 1 may be present in an amount of 5 to 30 mol%, based on the total moles of all lipids in the peptide-liposome complex.

The peptide-liposome complex may be a spherical hollow body having an average diameter of 50 to 300 nm and composed of a lipid bilayer membrane.

The peptide-liposome complex may further include an anticancer agent.

A further aspect of the present invention provides a composition for diagnosing cancer including a peptide-liposome complex and a fluorescent molecule.

The cancer may be derived from cancer cells overexpressing PD-L1 on the cell surface.

Another aspect of the present invention provides a pharmaceutical composition for preventing or treating cancer including a peptide-liposome complex.

The cancer may be selected from the group consisting of gastric cancer, lung cancer, non-small cell lung cancer, breast cancer, ovarian cancer, liver cancer, bronchial cancer, nasopharyngeal cancer, laryngeal cancer, pancreatic cancer, bladder cancer, colon cancer, cervical cancer, bone cancer, non-small cell bone cancer, hematologic malignancy, skin cancer, head or neck cancer, uterine cancer, rectal cancer, perianal cancer, fallopian tube cancer, endometrial cancer, vaginal cancer, vulvar cancer, Hodgkin’s disease, esophageal cancer, small intestine cancer, endocrine gland cancer, thyroid cancer, parathyroid cancer, adrenal cancer, soft tissue sarcoma, urethral cancer, penile cancer, prostate cancer, chronic or acute leukemia, lymphocytic lymphoma, kidney or ureter cancer, renal cell carcinoma, renal pelvic carcinoma, salivary gland cancer, sarcoma, pseudomyxoma, hepatoblastoma, testicular cancer, glioblastoma, lip cancer, ovarian germ cell tumor, basal cell carcinoma, multiple myeloma, gallbladder cancer, choroidal melanoma, ampulla of Vater cancer, peritoneal cancer, tongue cancer, small cell cancer, pediatric lymphoma, neuroblastoma, duodenal cancer, ureteral cancer, astrocytoma, meningioma, renal pelvis cancer, vulvar cancer, thymus cancer, central nervous system (CNS) tumor, primary central nervous system lymphoma, spinal cord tumor, brainstem glioma, pituitary adenoma, and combinations thereof.

The peptide-liposome complex of the present invention is optimal for multivalent crosslinking with PD-L1. The peptide-liposome complex of the present invention effectively blocks PD-L1, an immune checkpoint on the surface of cancer cells, and prevents the recycling of PD-L1 by intracellular metabolism to induce complete degradation of PD-L1 in cancer cells. In practice, the peptide-liposome complex of the present invention was verified to have an increased therapeutic effect on cancer, enabling more fundamental cancer treatment.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a schematic diagram showing the working principle of a peptide-liposome complex according to the present invention;

FIG. 2 shows a lipid conjugate (DPSE-PEG2000-PD-L1) consisting of a second phospholipid and a peptide having the amino acid sequence set forth in SEQ ID NO: 1 and a ¹H NMR spectrum of the lipid conjugate;

FIG. 3 shows the results of stability evaluation of particles of peptide-liposome complexes prepared in Examples 1 to 3;

FIG. 4 shows the results of cytotoxicity evaluation of peptide-liposome complexes prepared in Examples 1 to 3;

FIG. 5 shows confocal microscopy images of trans-CT26 cells treated with liposomes of Comparative Example 1 and peptide-liposome complexes prepared in Examples 1 to 3;

FIG. 6 shows fluorescence images revealing the expression profiles of PD-L1 (green fluorescence) in trans-CT26 cells treated with a peptide-liposome complex (10-PD-L1 Lipo) prepared in Example 2 and trans-CT26 cells treated with PD-L1 monoclonal antibody (aPD-L1 antibody);

FIG. 7 shows the results of quantitative analysis of the expression levels of PD-L1 (green fluorescence) from the results of FIG. 6;

FIG. 8A shows fluorescence images revealing the expression profiles of lysosomes (blue fluorescence) in trans-CT26 cells treated with a peptide-liposome complex (10-PD-L1 Lipo) prepared in Example 2 and trans-CT26 cells treated with PD-L1 monoclonal antibody (aPD-L1 antibody) and FIG. 8B shows the results of quantitative analysis of the lysosome colocalization profiles for the red fluorescence measured in the fluorescence images of FIG. 8A;

FIG. 9 shows microscopy images of trans-CT26 cells treated with a peptide-liposome complex (10-PD-L1 Lipo) prepared in Example 2 and co-cultured with T cells and trans-CT26 cells treated with PD-L1 monoclonal antibody (aPD-L1 antibody) and co-cultured with T cells;

FIG. 10A shows proportions of dead cancer cells and FIG. 10B shows results of quantitative analysis of the concentrations of released interferon gamma (IFN-γ) after co-culture of trans-CT26 cells treated with a peptide-liposome complex (10-PD-L1 Lipo) prepared in Example 2 with T cells and after co-culture of trans-CT26 cells treated with PD-L1 monoclonal antibody (aPD-L1 antibody) with T cells;

FIG. 11 shows the results of in vivo fluorescence analysis for colorectal cancer animal models in Group 3 administered a peptide-liposome complex (10-PD-L1 Lipo) prepared in Example 2, Group 2 administered liposomes (PEG-Lipo) of Comparative Example 1, and non-treated Group 1;

FIG. 12 shows the results of fluorescence analysis for tumor tissues excised from animal models in Groups 1, 2 and 3;

FIG. 13 shows fluorescence microscopy images of tumor tissues excised from animal models in Groups 1, 2 and 3 after staining with anti-PD-L1 antibody;

FIG. 14A shows changes in tumor volume (V; mm³) in animal models in Groups 1, 2, and 3 during the treatment period and FIG. 14B shows changes in the weight of animal models in Groups 1, 2, and 3 during the treatment period;

FIG. 15 shows TUNEL-stained tumor tissues excised from animal models in Groups 1, 2, and 3 20 days after drug administration;

FIG. 16 shows the proportions of T cells expressing CD45, CD3, and CD8 in tumor tissues excised from animal models in Groups 1, 2, and 3 20 days after drug administration, which were analyzed by flow cytometry; and

FIG. 17 shows the proportions of regulatory T cells expressing CD3, CD4, and FoxP3 in tumor tissues excised from animal models in Groups 1, 2, and 3 20 days after drug administration, which were analyzed by flow cytometry.

### DETAILED DESCRIPTION OF THE INVENTION

The present invention will now be described in more detail.

The present inventors have discovered a peptide capable of binding to and degrading PD-L1 present on the surface of cancer cells. The present inventors have also discovered that when the peptide is bound to liposomes for inducing multivalent binding to PD-L1 as well as selective targeting efficiency to cancer, the peptide-liposome complex can prevent recycling of PD-L1 and induce the mechanism of complete degradation of PD-L1 in cells, achieving an enhanced immune response to cancer. Finally, the present inventors have elucidated the effects of the peptide-liposome complex and arrived at the present invention.

One aspect of the present invention is directed to a peptide-liposome complex composed of a lipid bilayer including (a) a first phospholipid, (b) a second phospholipid containing PEG, (c) cholesterol, and (d) a lipid conjugate consisting of the second phospholipid and a peptide having the amino acid sequence set forth in SEQ ID NO: 1.

The peptide-liposome complex of the present invention is composed of at least one lipid bilayer and may include an aqueous compartment enclosed by the lipid bilayer. When lipids containing hydrophilic head groups are dispersed in water, they can spontaneously form bilayer membranes, also called lamellae. Lamellae are composed of two monolayer sheets of lipid molecules with their nonpolar (hydrophobic) surfaces facing each other and their polar (hydrophilic) surfaces facing the aqueous medium. The peptide-liposome complex may include unilamellar vesicles composed of a single lipid bilayer and may generally have a diameter ranging from about 1 to about 1000 nm, about 10 to about 800 nm, or about 50 to 300 nm, more preferably 100 to 200 nm. Since the peptide-liposome complex has a uniform particle size distribution with a specific particle size, it can easily penetrate tissues at tumor sites where blood vessels are very weak and have loose structures. Thus, the peptide-liposome complex whose diameter is within the range defined above can be applied to tumor tissues throughout the body regardless of where it is administered.

The peptide-liposome complex has a zeta potential ranging from -15 to -10 mV. Within this range, the peptide-liposome complex can stably exist in solution without aggregation for a long period of time.

The peptide-liposome complex of the present invention may also be multilamellar, typically with a diameter in the range of 1 to 10 µm. The multilamellar peptide-liposome complex includes two to several hundred concentric lipid bilayers alternating with layers of an aqueous phase anywhere. The peptide-liposome complex may also include multilamellar vesicles (MLVs), large unilamellar vesicles (LUVs), and small unilamellar vesicles (SUVs). Each of the first phospholipid and the second phospholipid of the peptide-liposome complex may independently be selected from cationic phospholipids, zwitterionic phospholipids, neutral phospholipids, anionic phospholipids, and combinations thereof.

The peptide-liposome complex of the present invention may contain any suitable lipids, including cationic, zwitterionic, neutral or anionic lipids, as described above. Examples of the suitable lipids include fats, waxes, steroids, cholesterol, fat-soluble vitamins, monoglycerides, diglycerides, phospholipids, sphingolipids, glycolipids, cationic lipids, anionic lipids, and derivatized lipids.

The first phospholipid is not particularly limited and may be selected from the group consisting of phosphatidylcholine (PC), phosphatidic acid (PA), phosphatidylethanolamine (PE), phosphatidylglycerol (PG), phosphatidylserine (PS), phosphatidylinositol (PI), dimyristoylphosphatidylcholine (DMPC), distearoylphosphatidylcholine (DSPC), dioleoylphosphatidylcholine (DOPC), dipalmitoylphosphatidylcholine (DPPC), dimyristoylphosphatidylglycerol (DMPG), distearoylphosphatidylglycerol (DSPG), dioleoylphosphatidylglycerol (DOPG), dipalmitoylphosphatidylglycerol (DPPG), dimyristoylphosphatidylserine (DMPS), distearoylphosphatidylserine (DSPS), dioleoylphosphatidylserine (DOPS), dipalmitoylphosphatidylserine (DPPS), dioleoylphosphatidylethanolamine (DOPE), palmitoyloleoylphosphatidylcholine (POPC), palmitoyloleoylphosphatidylethanolamine (POPE), dioleoylphosphatidylethanolamine 4-(N-maleimidomethyl)-cyclohexane-1-carboxylate (DOPE-mal), dipalmitoylphosphatidylethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidylethanolamine (SOPE), 1,2-dielaidoyl-sn-glycero-3-phosphoethanolamine (transDOPE), cardiolipin, and mixtures thereof. The first phospholipid is preferably palmitoyloleoylphosphatidylcholine (POPC) as a neutral lipid. Neutral lipids can increase targeting efficiency to cancer cells due to their weak bonding strength to the cell surface.

The second phospholipid may be a derivatized lipid. For example, the second phospholipid may be selected from PEGylated lipids. Specifically, the second phospholipid may be 1,2-distearoyl-sn-glycero-3-phosphoethanolamine-N-[methoxy(polyethylene glycol)2000] (DSPE-mPEG2000) or 1,2-disteroyl-sn-glycero-3-phosphoethanolamine-N-[maleimide(polyethylene glycol)2000] (DSPE-PEG2000-MAL). The second phospholipid is preferably 1,2-distearoyl-sn-glycero-3-phosphoethanolamine-N-[methoxy(polyethylene glycol)2000], more preferably DSPE-mPEG2000 having a molecular weight in the range of 2650 to 3050. The second phospholipid serves to enhance the hydrophilicity of the peptide-liposome complex, impart structural stability to the peptide-liposome complex, increase the intracellular or in vivo turnover time of the peptide-liposome complex, and prevent disappearance of the peptide-liposome complex by the immune system.

The first and second phospholipids of the peptide-liposome complex according to the present invention may be selected depending on desired characteristics such as leakage rate, stability, particle size, zeta potential, protein binding, in vivo circulation, and/or accumulation in tissues or organs. Preferably, the first phospholipid is POPC and the second phospholipid containing PEG is DSPE-PEG(2000).

The lipid conjugate is a linear conjugate formed by binding the second phospholipid with a peptide having the amino acid sequence set forth in SEQ ID NO: 1:

Asn-Tyr-Ser-Lys-Pro-Thr-Asp-Arg-Gln-Tyr-His-Phe (1)

The peptide may be in D- or L-form as determined from its whole sequence. The peptide is preferably in D-form.

A variant or fragment of the peptide having the amino acid sequence set forth in SEQ ID NO: 1 in which one or more of the amino acid residues are deleted, inserted, and/or substituted, may be used as long as it does not affect the structure and activity of the peptide-liposome complex according to the present invention. Exchanges of amino acids in proteins or peptides that do not entirely alter the activity of the molecules are known in the art. In some cases, the peptide may be modified by phosphorylation, sulfidation, acrylation, glycosylation, methylation, famesylation, etc. The amino acid sequence of the modified peptide may have an identity of 70, 80, 85, 90, 95 or 98% to the amino acid sequence set forth in SEQ ID NO: 1.

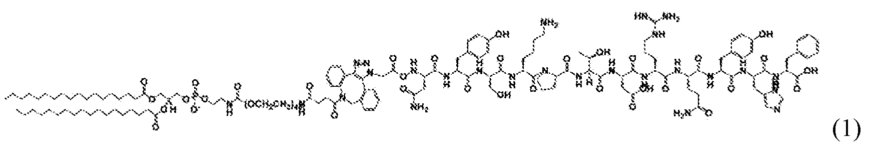 [SMILES: C=C(C)C1Cc2ccccc2/C=C\c2ccccc21]

The second phospholipid is directly bound to the N-terminus of the peptide having the sequence set forth in SEQ ID NO: 1. Alternatively, the second phospholipid may be bound to the N-terminus of the peptide via a linker.

The linker connects the N-terminus of the peptide and the second phospholipid. It is important that the linker is linked to the N-terminus of the peptide. Only this linkage allows the peptide-liposome complex to effectively interact with PD-L1 present on the surface of cancer cells even in vivo. If the linker is linked to the C-terminus of the peptide, the sequence of the peptide cannot bind to PD-L1 present on the surface of cancer cells.

The linker may be formed by bioorthogonal click chemistry with an azido group.

Specifically, an azido group may be present at the N-terminus of the peptide. The azido group is preferably azidoacetyl.

A cycloalkyne group may be introduced into the second phospholipid such that it forms a bond by bioorthogonal click chemistry with the azido group introduced at the N-terminus of the peptide.

The cycloalkyne group may be selected from those represented by Formulae 2 to 6:

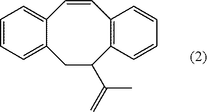 [SMILES: OC1Cc2ccccc2C#Cc2ccccc21]

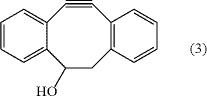 [SMILES: O=C1c2ccccc2C#Cc2ccccc2N1CC1CC(c2ccc(CO)cc2)=NO1]

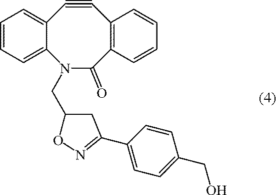 [SMILES: O=C(O)CCCC(=O)N1Cc2ccccc2C#Cc2ccccc21]

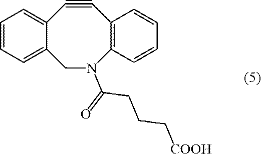

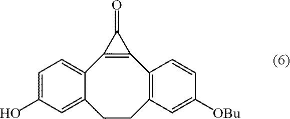

Specifically, the lipid conjugate consisting of the second phospholipid and the peptide having the amino acid sequence set forth in SEQ ID NO: 1 is present in an amount of about 0 mol% to about 90 mol%, about 1 mol% to about 70 mol%, about 5 mol% to about 50 mol%, about 5 mol% to about 30 mol% or about 10 mol% to about 15 mol%, based on the total moles of all lipids in the peptide-liposome complex.

The peptide-liposome complex of the present invention includes the first phospholipid (a), the second phospholipid (b), the cholesterol (c), and the lipid conjugate (d) in a molar ratio of 1.5-3:0-0.3:1:0.2-1.5, preferably in a molar ratio of 2.5-3.0:0.01-0.05:1:0.3-0.8, more preferably in a molar ratio of 2.8-2.9:0.04-0.05:1:0.4-0.5, even more preferably in a molar ratio of 66:1:23:10.

According to the most preferred embodiment of the present invention, the peptide-liposome complex includes 66 mol% of POPC, 23 mol% of cholesterol, 1 mol% of DSPE-PEG(2000), and 10 mol% of DSPE-PEG-PD-L1.

The peptide-liposome complex may be a spherical hollow body composed of a lipid bilayer including the first phospholipid (a), the second phospholipid containing PEG (b), the cholesterol (c), and the lipid conjugate consisting of the second phospholipid and the peptide having the amino acid sequence set forth in SEQ ID NO: 1 (d).

The peptide-liposome complex may further include a drug to induce a direct killing effect on cancer cells. The drug may be loaded in the peptide-liposome complex for delivery. The drug may be selected from the group consisting of anticancer agents, contrast agents (dyes), hormones, antihormones, vitamins, calcium agents, inorganic agents, saccharides, organic acid preparations, protein amino acid preparations, antidotes, enzymes, metabolic preparations, concomitant agents for diabetes, tissue growth stimulants, chlorophyll agents, pigment agents, anti-tumor agents, therapeutic agents for tumors, radiopharmaceuticals, tissue cell diagnostic agents, tissue cell therapeutic agents, antibiotic agents, antiviral agents, complex antibiotics, chemotherapeutic agents, vaccines, toxins, toxoids, antitoxins, leptospira serum, blood products, biological agents, analgesics, immunogenic molecules, antihistamines, anti-allergy medications, non-specific immunogenic agents, anesthetics, stimulants, psychotropic agents, small-molecule compounds, nucleic acids, aptamers, antisense nucleic acids, oligonucleotides, peptides, siRNAs, microRNAs, and mixtures thereof. The drug is preferably an anticancer agent.

The anticancer agent may be selected from the group consisting of camptothecin, doxorubicin, cisplatin, verapamil, fluorouracil, oxaliplatin, daunorubicin, irinotecan, topotecan, paclitaxel, carboplatin, gemcitabine, methotrexate, docetaxel, acivicin, aclarubicin, acodazole, acronycine, adozelesin, alanosine, aldesleukin, allopurinol sodium, altretamine, aminoglutethimide, amonafide, ampligen, amsacrine, androgens, anguidine, aphidicolin glycinate, asaley, asparaginase, 5-azacytidine, azathioprine, Bacillus Calmette-Guérin (BCG), Bacillus Calmette-Guérin (BCG), Baker’s antifol, beta-2-deoxythioguanosine, bisantrene HCl, bleomycin sulfate, busulfan, buthionine sulfoximine, BWA 773U82, BW 502U83/HCl, BW 7U85 mesylate, ceracemide, carbetimer, carboplatin, carmustine, chlorambucil, chloroquinoxaline-sulfonamide, chlorozotocin, chromomycin A3, cisplatin, cladribine, corticosteroids, Corynebacterium parvum, CPT-11, crisnatol, cyclocytidine, cyclophosphamide, cytarabine, cytembena, Dabis maleate, dacarbazine, dactinomycin, daunorubicin HCl, deazauridine, dexrazoxane, dianhydrogalactitol, diaziquone, dibromodulcitol, didemnin B, diethyldithiocarbamate, diglycoaldehyde, dihydro-5-azacytidine, echinomycin, edatrexate, edelfosine, eflornithine, Elliott’s solution, elsamitrucin, epirubicin, esorubicin, estramustine phosphate, estrogen, etanidazole, ethiofos, etoposide, fadrozole, fazarabine, fenretinide, filgrastim, finasteride, flavone acetic acid, floxuridine, fludarabine phosphate, 5-fluorouracil, Fluosol, flutamide, gallium nitrate, gemcitabine, goserelin acetate, hepsulfam, hexamethylene bisacetamide, homoharringtonine, hydrazine sulfate, 4-hydroxyandrostenedione, hydroxyurea, idarubicin HCl, ifosfamide, interferon alpha, interferon beta, interferon gamma, interleukin-1 alpha and beta, interleukin-3, interleukin-4, interleukin-6, 4-ipomeanol, iproplatin, isotretinoin, leucovorin calcium, leuprolide acetate, levamisole, liposomal daunorubicin, liposome-encapsulated doxorubicin, lomustine, lonidamine, maytansine, mechlorethamine hydrochloride, melphalan, menogaril, merbarone, 6-mercaptopurine, mesna, methanol extract of Bacillus Calmette-Guérin, methotrexate, N-methylformamide, mifepristone, mitoguazone, mitomycin-C, mitotane, mitoxantrone hydrochloride, monocyte/macrophage colony-stimulating factor, nabilone, nafoxidine, neocarzinostatin, octreotide acetate, ormaplatin, oxaliplatin, paclitaxel, PALA, pentostatin, piperazinedione, pipobroman, pirarubicin, piritrexim, piroxantrone hydrochloride, PIXY-321, plicamycin, porfimer sodium, prednimustine, procarbazine, progestins, pyrazofurin, razoxane, sargramostim, semustine, spirogermanium, spiromustine, streptonigrin, streptozocin, sulofenur, suramin sodium, tamoxifen, taxotere, tegafur, teniposide, terephthalamidine, teroxirone, thioguanine, thiotepa, thymidine injection, tiazofurin, topotecan, toremifene, tretinoin, trifluoperazine hydrochloride, trifluridine, trimetrexate, tumor necrosis factor (TNF), uracil mustard, vinblastine sulfate, vincristine sulfate, vindesine, vinorelbine, vinzolidine, Yoshi 864, zorubicin, pharmaceutically acceptable salts thereof, and mixtures thereof. The anticancer agent is preferably doxorubicin.

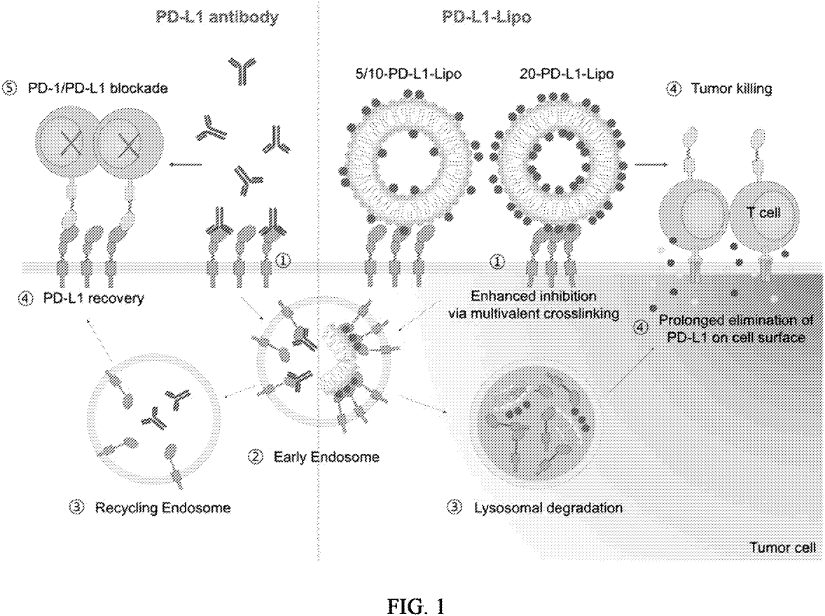

The peptide-liposome complex can specifically form multivalent bonds with PD-L1 present on the surface of cancer cells (FIG. 1). The cancer cells may be selected from the group consisting of gastric cancer, lung cancer, non-small cell lung cancer, breast cancer, ovarian cancer, liver cancer, bronchial cancer, nasopharyngeal cancer, laryngeal cancer, pancreatic cancer, bladder cancer, colon cancer, cervical cancer, bone cancer, non-small cell bone cancer, hematologic malignancy, skin cancer, head or neck cancer, uterine cancer, rectal cancer, perianal cancer, fallopian tube cancer, endometrial cancer, vaginal cancer, vulvar cancer, Hodgkin’s disease, esophageal cancer, small intestine cancer, endocrine gland cancer, thyroid cancer, parathyroid cancer, adrenal cancer, soft tissue sarcoma, urethral cancer, penile cancer, prostate cancer, chronic or acute leukemia, lymphocytic lymphoma, kidney or ureter cancer, renal cell carcinoma, renal pelvic carcinoma, salivary gland cancer, sarcoma, pseudomyxoma, hepatoblastoma, testicular cancer, glioblastoma, lip cancer, ovarian germ cell tumor, basal cell carcinoma, multiple myeloma, gallbladder cancer, choroidal melanoma, ampulla of Vater cancer, peritoneal cancer, tongue cancer, small cell cancer, pediatric lymphoma, neuroblastoma, duodenal cancer, ureteral cancer, astrocytoma, meningioma, renal pelvis cancer, vulvar cancer, thymus cancer, central nervous system (CNS) tumor, primary central nervous system lymphoma, spinal cord tumor, brainstem glioma, pituitary adenoma, and combinations thereof.

The peptide-liposome complex can specifically form multivalent bonds with PD-L1 present on the surface of cancer cells. Due to this ability, the peptide-liposome complex induces PD-L1 to the lysosomal pathway to prevent recycling of PD-L1, achieving complete degradation of PD-L1 (FIG. 1).

The peptide-liposome complex may be prepared by a thin-film hydration method using the first phospholipid (a), the second phospholipid containing PEG (b), the cholesterol (c), and the lipid conjugate consisting of the second phospholipid and the peptide having the amino acid sequence set forth in SEQ ID NO: 1 (d).

The peptide-liposome complex includes the first phospholipid (a), the second phospholipid (b), the cholesterol (c), and the lipid conjugate (d) in a molar ratio of 1.5-3:0-0.3:1:0.2-1.5, preferably in a molar ratio of 2.5-3.0:0.01-0.05:1:0.3-0.8, more preferably in a molar ratio of 2.8-2.9:0.04-0.05: 1:0.4-0.5, even more preferably in a molar ratio of 66:1:23:10.

According to the most preferred embodiment of the present invention, the peptide-liposome complex includes 66 mol% of POPC, 23 mol% of cholesterol, 1 mol% of DSPE-PEG(2000), and 10 mol% of DSPE-PEG-PD-L1.

The peptide-liposome complex may be prepared in the form of giant single-walled vesicles by pulverizing during repeated freezing and thawing. The freezing and thawing may be performed for a total of 5 to 30 cycles.

The peptide-liposome complex may be homogenized by filtration. The filtration is not particularly limited and may be performed by any suitable method known in the art.

A further aspect of the present invention is directed to a composition for diagnosing cancer including the peptide-liposome complex and a fluorescent molecule.

Another aspect of the present invention is directed to a pharmaceutical composition for preventing or treating cancer including the peptide-liposome complex.

In the composition of the present invention, a fluorescent molecule may be inserted into the lipid bilayer of the peptide-liposome complex or loaded in the hollow of the peptide-liposome complex. The use of the peptide-liposome complex enables cancer cell-specific delivery of the fluorescent molecule and fluorescence imaging of cancer cells. Accordingly, the presence of the peptide-liposome complex makes the composition suitable for use in in vivo imaging of cancer cells as well as cancer diagnosis.

The fluorescent molecule may be selected from the group consisting of, but not particularly limited to, Cy3, Cy5, poly L-lysine-fluorescein isothiocyanate (FITC), rhodamine-B-isothiocyanate (RITC), and rhodamine molecules.

As used herein, the term “prevent”, “preventing” or “prevention” means all actions that inhibit or delay the onset, progression or recurrence of the cancer disease by administration of the composition according to the present invention. As used herein, the term “treat”, “treating” or “treatment” means all actions that improve or beneficially change symptoms of the cancer disease by administration of the composition according to the present invention.

As used herein, the term “diagnose”, “diagnosing” or diagnosis” is intended to include determining the susceptibility of an object to a particular disease or disorder, determining whether an object currently has a particular disease or disorder, determining the prognosis of an object with a particular disease or disorder, or therametrics (e.g., monitoring the condition of the object to provide information on treatment efficacy).

As used herein, the term “pharmaceutical composition” refers to a composition that is prepared for preventing or treating a disease. The pharmaceutical composition may be formulated into various preparations by suitable methods known in the art. Examples of such preparations include oral preparations such as powders, granules, tablets, capsules, suspensions, emulsions, and syrups and other preparations such as external preparations, suppositories, and sterile injectable solutions.

As used herein, the term “including as an active ingredient” means that the corresponding ingredient is present in an amount necessary or sufficient to achieve the desired biological effect. In practical applications, the amount of the active ingredient is determined as an amount for treating a target disease without causing other toxicities. For example, the amount of the active ingredient may vary depending on various factors such as the disease or condition to be treated, the form of the composition to be administered, the size of a subject, and the severity of the disease or condition. One of ordinary skill in the art to which the present invention pertains can empirically determine the effective amount of the composition without undue experimentation.

A pharmaceutically effective amount of the composition according to the present invention is administered orally or parenterally according to the desired method. As used herein, the term “pharmaceutically effective amount” refers to an amount sufficient to treat diseases at a reasonable benefit/risk ratio applicable to any medical treatment. The effective dosage level of the composition may be determined depending on factors, including the general health of the patient, the severity of the disease, the activity of the drug, the sensitivity to the drug, the time and route of administration, the rate of excretion, the duration of treatment, and the type of compounded or concurrent drugs, and other factors well-known in the medical field.

Therefore, the pharmaceutical composition of the present invention can be administered to an individual to prevent, treat, and/or diagnose tumor. As used herein, the term “tumor” is intended to include all pre-cancerous cells and cancer cells that exhibit neoplastic cell growth and proliferation, irrespective of whether they are malignant or benign. The cancer may be selected from the group consisting of, but not limited to, gastric cancer, lung cancer, non-small cell lung cancer, breast cancer, ovarian cancer, liver cancer, bronchial cancer, nasopharyngeal cancer, laryngeal cancer, pancreatic cancer, bladder cancer, colon cancer, cervical cancer, bone cancer, non-small cell bone cancer, hematologic malignancy, skin cancer, head or neck cancer, uterine cancer, rectal cancer, perianal cancer, fallopian tube cancer, endometrial cancer, vaginal cancer, vulvar cancer, Hodgkin’s disease, esophageal cancer, small intestine cancer, endocrine gland cancer, thyroid cancer, parathyroid cancer, adrenal cancer, soft tissue sarcoma, urethral cancer, penile cancer, prostate cancer, chronic or acute leukemia, lymphocytic lymphoma, kidney or ureter cancer, renal cell carcinoma, renal pelvic carcinoma, salivary gland cancer, sarcoma, pseudomyxoma, hepatoblastoma, testicular cancer, glioblastoma, lip cancer, ovarian germ cell tumor, basal cell carcinoma, multiple myeloma, gallbladder cancer, choroidal melanoma, ampulla of Vater cancer, peritoneal cancer, tongue cancer, small cell cancer, pediatric lymphoma, neuroblastoma, duodenal cancer, ureteral cancer, astrocytoma, meningioma, renal pelvis cancer, vulvar cancer, thymus cancer, central nervous system (CNS) tumor, primary central nervous system lymphoma, spinal cord tumor, brainstem glioma, pituitary adenoma, and combinations thereof.

As used herein, the term “individual” is meant to include mammals such as rats, livestock, mice, and humans. The individual is preferably a human.

The pharmaceutical composition of the present invention may be formulated into various preparations for administration to individuals. A representative preparation for parenteral administration is an injectable preparation, preferably an isotonic aqueous solution or suspension. The injectable preparation may be prepared using a suitable dispersant or wetting agent and a suitable suspending agent by a suitable technique known in the art. For example, a solution of ingredients in saline or buffer may be formulated into an injectable preparation. The pharmaceutical composition of the present invention may also be formulated into preparations for oral administration. Examples of the preparations for oral administration include ingestible tablets, buccal tablets, troches, capsules, elixirs, suspensions, syrups, and wafers. These preparations may include diluents (e.g., lactose, dextrose, sucrose, mannitol, sorbitol, cellulose, and/or glycine) and lubricants (e.g., silica, talc, stearic acid and its magnesium or calcium salt, and/or polyethylene glycol), in addition to the active ingredient. The tablets may include binders such as magnesium aluminum silicate, starch paste, gelatin, tragacanth, methylcellulose, sodium carboxymethylcellulose, and/or polyvinylpyrrolidine, and optionally disintegrating agent such as starch, agar, alginic acid or its sodium salt, absorbents, colorants, flavoring agents, and/or sweetening agents. The preparations may be prepared by known techniques such as mixing, granulation or coating.

The pharmaceutical composition of the present invention may further include an adjuvant such as a preservative, a hydrating agent, an emulsification accelerator, or a salt or buffer for osmotic pressure control and other therapeutically useful substances. In this case, the pharmaceutical composition may be formulated by a suitable method known in the art.

The pharmaceutical composition of the present invention may be administered via various routes, for example, orally, transdermally, subcutaneously, intravenously or intramuscularly, preferably intravenously. The dose of the pharmaceutical composition according to the present invention may be appropriately selected according to various factors such as the route of administration, the patient’s age, sex, and weight, and the severity of the disease. The composition of the present invention may be also administered in parallel with a known compound capable of enhancing the desired effect.

The present invention will be specifically explained with reference to the following examples, including experimental examples. However, these examples are merely illustrative and are not intended to limit the scope of the present invention.

### EXAMPLES

### Experimental Materials

The peptide (Azidoacetyl-nAsn-Tyr-Ser-Lys-Pro-Thr-Asp-Arg-Gln-Tyr-His-Phe) was purchased from Peptron Inc. (Daejeon, Korea). 1,2-Distearoyl-sn-glycero-3-phosphoethanolamine-N-[amino(polyethylene glycol)-2000]-dibenzocyclooctyl (DSPE-PEG2000-DBCO), 1,2-distearoyl-sn-glycero-3-phosphoethanolamine-N-[amino(polyethylene glycol)-2000] (DSPE-PEG2000) (ammonium salt), 1-palmitoyl-2-oleoyl-sn-glycero-3-phosphocholine (POPC), and cholesterol were purchased from Merck (Germany). Ethanol (EtOH), chloroform, and dimethyl sulfoxide (DMSO) were purchased from Daejung Chemical (Korea). PD-L1 antibody, CD45 T cell antibody, CD3 T cell antibody, CD8 T cell antibody, CD4 T cell antibody, and Foxp3 antibody were purchased from BioLegend (USA). RPMI 1640 media, antibiotics, and fetal bovine serum were purchased from SciLab Korea (Seoul, Korea). CT26 was purchased from the American Type Culture Collection (USA) and Balb/c mice were purchased from Narabio (Daejeon, Korea).

### Preparative Example 1. Synthesis of DPSE-PEG2000-PD-L1 (Lipid Conjugate)

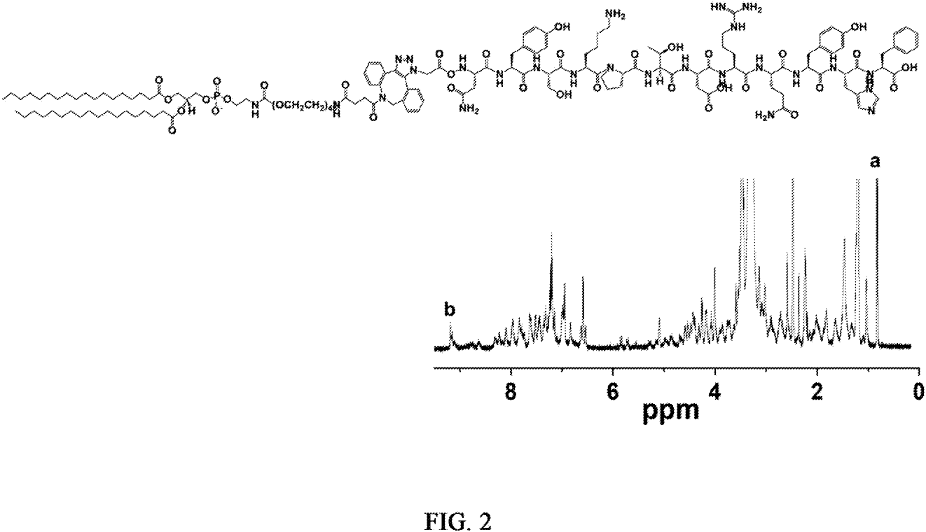

8 mg of 1,2-distearoyl-sn-glycero-3-phosphoethanolamine-N-[amino(polyethylene glycol)-2000]-dibenzocyclooctyl (DSPE-PEG2000-DBCO) was dissolved in 800 µl of DMSO to prepare a DPSE-PEG2000 solution. 20 mg of the peptide having an azido group and the amino acid sequence set forth in SEQ ID NO: 1 (azidoacetyl-nAsn-Tyr-Ser-Lys-Pro-Thr-Asp-Arg-Gln-Tyr-His-Phe) (D form) was dissolved in 200 µl of distilled water to prepare a peptide solution. The DPSE-PEG2000 solution was mixed with the peptide solution and the mixture was allowed to react at 37° C. for 24 h to obtain a lipid conjugate (DPSE-PEG2000-PD-L1) consisting of the second phospholipid and the peptide having the amino acid sequence set forth in SEQ ID NO: 1. The chemical structure of PD-L1-PEG was analyzed by nuclear magnetic resonance (NMR) spectroscopy. The results are shown in FIG. 2.

### Comparative Example 1 and Examples 1-4. Preparation of PD-L1 Lipo

Each of DSPE-PEG2000, POPC, and cholesterol was dissolved to 100 mg/mL in chloroform. DPSE-PEG2000-PD-L1 synthesized in Preparative Example 1 was dissolved to 10 mg/mL in a mixed solvent of ethanol and chloroform (1:1). The resulting solutions were mixed in the molar ratios shown in Table 1. The solvents were removed from each mixed solution using a rotary evaporator for 10 min. The residue was dispersed in phosphate buffered saline (PBS, pH 7) at 50° C. for 30 min. Then, the dispersion was ultrasonicated for 2 min and filtered through a 0.2 nm filter and an extruder to obtain a peptide-liposome complex.

The mixing molar ratios of DSPE-PEG2000, POPC, cholesterol, and DPSE-PEG2000-PD-L1 for preparing the peptide-liposome complex are shown in Table 1.

TABLE 1

Molar ratio

| | First phospholipid POPC | Second phospholipid DSPE-PEG200 | Cholesterol | Lipid conjugate DPSE-PEG2000-PD-L1 |
|---|---|---|---|---|
| Comparative Example 1 (PEG-Lipo) | 66 | 11 | 23 | 0 |
| Example 1 (5-PD-L1 Lipo) | 66 | 6 | 23 | 5 |
| Example 2 (10-PD-L1 Lipo) | 66 | 1 | 23 | 10 |
| Example 3 (20-PD-L1 Lipo) | 57 | 0 | 23 | 20 |
| Example 4 (30-PD-L1 Lipo) | 47 | 0 | 23 | 30 |

### Experimental Example 1. Analysis of Sizes and Stabilities of the Peptide-Liposome Complexes

The sizes and stabilities of the peptide-liposome complexes prepared in Examples 1-4 were analyzed. Specifically, 1.0 mg of each of the peptide-liposome complexes prepared in Examples 1-4 was dispersed in 1 mL of PBS (pH 7) and the particle diameter and size distribution of the peptide-liposome complex were measured using a Zetasizer (NanoZS, Malvern, U.K.). Changes in particle size for 6 days were analyzed to evaluate the stability of the peptide-liposome complex.

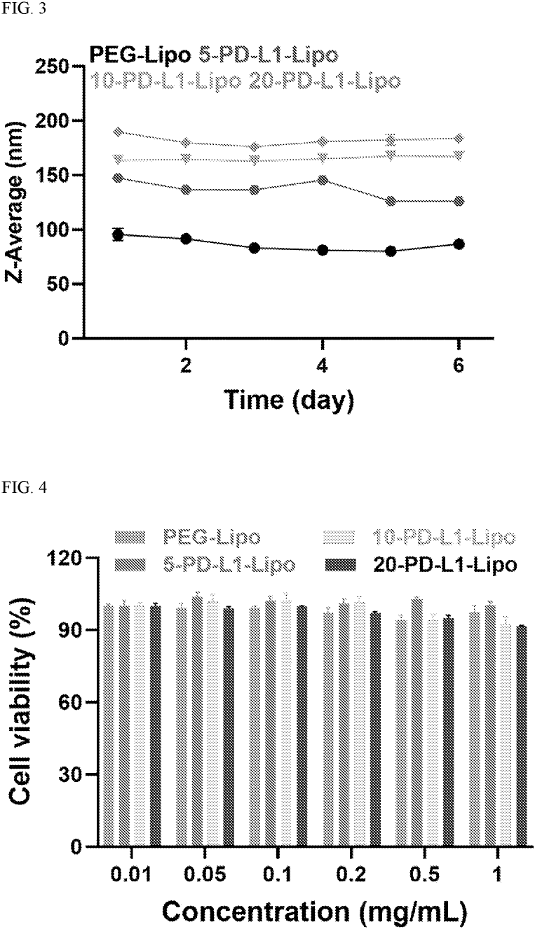

The particle sizes and surface potentials of the peptide-liposome complexes prepared in Examples 1-3 are shown in Table 2. FIG. 3 shows the results of stability evaluation of the particles of the peptide-liposome complexes prepared in Examples 1-3.

TABLE 2

| | Diameter (nm) | Zeta potential (mV) |
|---|---|---|
| Comparative Example 1 (PEG-Lipo) | 95.7 ± 2.9 | -17.2 ± 5.09 |
| Example 1 (5-PD-L1 Lipo) | 115.0 ± 0.98 | -13.3 ± 5.66 |
| Example 2 (10-PD-L1 Lipo) | 163.7 ± 1.65 | -12.2 ± 4.33 |
| Example 3 (20-PD-L1 Lipo) | 189.6 ± 3.11 | -10.8 ± 4.24 |

As shown in Table 2, the peptide-liposome complexes prepared in Examples 1-3 had sizes of 50-300 nm.

As shown in FIG. 3, the peptide-liposome complexes prepared in Examples 1-3 were maintained stable without changes in particle size for 6 days.

### Experimental Example 2. Cytotoxicities of the Peptide-Liposome Complexes

An in vitro cell experiment was conducted using colorectal cancer cells CT26 to determine whether the peptide-liposome complexes prepared in Examples 1-3 had negative effects on the cells. First, CT26 cells were plated in a 96-well cell culture plate at a density of 2 × 10⁴ cells/well. DMEM supplemented with 10% (v/v) fetal bovine serum (FBS) and 1% penicillin-streptomycin were used as culture media. After stabilization in a humid environment of 5% CO₂ and 95% air at 37° C. for 24 h, each cell culture medium was treated with various concentrations (0.01 mg/ml, 0.05 mg/ml, 0.1 mg/ml, 0.2 mg/ml, 0.5 mg/ml, and 1 mg/ml) of the peptide-liposome complexes prepared in Examples 1-3 and cells were cultured in an incubator at 37° C. for 48 h. After completion of the culture, each well was treated with 10 µg of cell counting kit-8 (CCK-8) solution and incubated for 30 min. The absorbance at 450 nm was measured using a microplate reader (VERSAmax™, Molecular Devices Corp., Sunnyvale, CA).

FIG. 4 shows the results of cytotoxicity evaluation of the liposomes of Comparative Example 1 and the peptide-liposome complexes prepared in Examples 1-3. As shown in FIG. 4, the peptide-liposome complexes prepared in Examples 1-3 caused no significant toxicities in cells, suggesting that they are stable materials.

### Experimental Example 3. Evaluation of Binding Capacities of the Peptide-Liposome Complexes to PD-L1

An investigation was made as to whether the peptide-liposome complexes prepared in Examples 1-3 were specifically bound to PD-L1 on the cell surface.

CT26 cancer cells were plated in a cell culture plate for confocal microscopy at a density of 2 × 10⁴ cells/well. After stabilization for 24 h, cells were treated with the liposomes of Comparative Example 1 and the peptide-liposome complexes prepared from Examples 1-3 (each 0.5 mg/mL) and cultured at 4° C. for 1 h. After completion of the culture, cells were treated with a fixative for 15 min, treated with a DAPI solution for 10 min to stain nuclei, and analyzed by confocal fluorescence microscopy. PD-L1 expressing GFP (green fluorescence) by transfection was allowed to be expressed on the CT26 cancer cells (“trans-CT26”).

FIG. 5 shows confocal microscopy images of the trans-CT26 cells treated with liposomes of Comparative Example 1 and the peptide-liposome complexes prepared in Examples 1-3.

As shown in FIG. 5, PD-L1 present on the surface of the trans-CT26 cells was identified in green fluorescence, the nuclei of the trans-CT26 cells were identified in blue fluorescence, and the peptide-liposome complexes were identified in red fluorescence.

These results demonstrated that the peptide-liposome complexes prepared in Examples 1-3 were all successfully bound to PD-L1 present on the surface of the trans-CT26 cells. Particularly, the peptide-liposome complex prepared in Example 2 was significantly bound to PD-L1 with the highest efficiency.

The liposomes of Comparative Example 1 hardly formed bonds with PD-L1 present on the surface of the trans-CT26 cells.

That is, the presence of DSPE-PEG2000-PD-L1 in an amount of less than 5 mol% with respect to the total moles of all lipids in the peptide-liposome complex led to a significant reduction in the ability to recognize PD-L1. Meanwhile, DSPE-PEG2000-PD-L1 present in an amount of more than 20 mol% sterically interfered with the binding of the peptide-liposome complex to PD-L1 on the cell surface, resulting in a decrease in binding efficiency.

### Experimental Example 4. PD-L1 Degradation by the Peptide-Liposome Complex

The mechanism of PD-L1 degradation by the peptide-liposome complex prepared in Example 2 in cells was investigated.

### 4-1. Confocal Fluorescence Microscopy

First, trans-CT26 cancer cells were plated in a cell culture plate for confocal microscopy at a density of 2 × 10⁴ cells/well. After stabilization for 24 h, cells were treated with PD-L1 monoclonal antibody (PD-L1 antibody) and the peptide-liposome complex prepared in Example 2 (each 0.5 mg/mL) and cultured at 37° C. for various times (0, 3, 6, 12, and 24 hours). Cells were treated with a fixative for 15 min, treated with a DAPI solution for 10 min to stain nuclei, and analyzed by confocal fluorescence microscopy.

### 4-2. Lysosomes

Next, an investigation was made as to whether the peptide-liposome complex prepared in Example 2 was bound to PD-L1 via multivalent crosslinking and entered lysosomes in cells for PD-L1 degradation. To this end, trans-CT26 cancer cells were placed in a cell culture plate for confocal microscopy at a density of 2 × 10⁴ cells/well. After stabilization for 24 h, cells were treated with PD-L1 monoclonal antibody (aPD-L1 antibody) and the peptide-liposome complex prepared in Example 2 (each 0.5 mg/mL) and cultured at 37° C. for 6 h. After completion of the culture, lysosomes in cells were stained with Lysotracker for 1 h. Cells were treated with a fixative for 15 min, treated with a DAPI solution for 10 min to stain nuclei, and analyzed by confocal fluorescence microscopy.

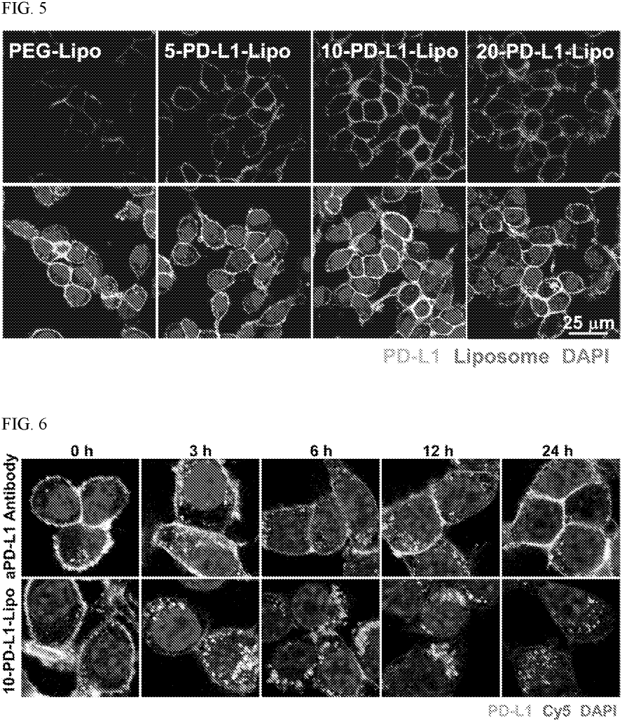

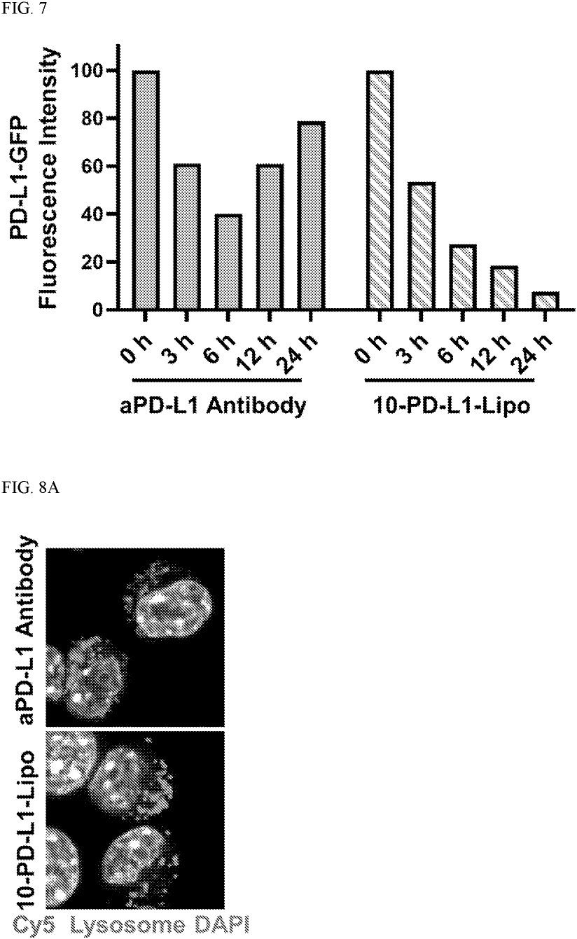

FIG. 6 shows fluorescence images revealing the expression profiles of PD-L1 (green fluorescence) in the trans-CT26 cells treated with the peptide-liposome complex (10-PD-L1 Lipo) prepared in Example 2 and the trans-CT26 cells treated with PD-L1 monoclonal antibody (aPD-L1 antibody). FIG. 7 shows the results of quantitative analysis of the expression levels of PD-L1 (green fluorescence) from the results of FIG. 6.

As shown in FIGS. 6 and 7, PD-L1 (green fluorescence) present on the surface of the trans-CT26 cells treated with the peptide-liposome complex (10-PD-L1 Lipo) prepared in Example 2 was decreased significantly as the drug treatment time increased. More than 90% of PD-L1 was completely degraded in the cells treated with the peptide-liposome complex (10-PD-L1 Lipo) prepared in Example 2.

In contrast, PD-L1 (green fluorescence) present on the surface of the trans-CT26 cells treated with PD-L1 monoclonal antibody (aPD-L1 antibody) was decreased up to 6 h, and thereafter, it was increased and restored to its initial level.

In summary, when cells were treated with PD-L1 monoclonal antibody (aPD-L1 antibody), PD-L1 was recycled without being degraded and again exposed to the cell surface. However, the treatment with the peptide-liposome complex (10-PD-L1 Lipo) prepared in Example 2 prevented the recycling of PD-L1 and led to complete degradation of PD-L1.

FIG. 8A shows fluorescence images revealing the expression profiles of lysosomes (blue fluorescence) in the trans-CT26 cells treated with the peptide-liposome complex (10-PD-L1 Lipo) prepared in Example 2 and the trans-CT26 cells treated with PD-L1 monoclonal antibody (aPD-L1 antibody) and FIG. 8B shows the results of quantitative analysis of the lysosome colocalization profiles for the red fluorescence measured in the fluorescence images of FIG. 8A.

As shown in FIGS. 8A and 8B, ~60% of the peptide-liposome complex (10-PD-L1 Lipo) (red fluorescence; Cy5) prepared in Example 2 was located in the lysosomes (green fluorescence) of the trans-CT26 cells.

In contrast, ~20% of the PD-L1 monoclonal antibody (aPD-L1 antibody) was located in the lysosomes (green fluorescence) of the trans-CT26 cells.

That is, the peptide-liposome complex prepared in Example 2 was bound to PD-L1 on the cell surface via multivalent crosslinking and then entered the lysosomes of the cells to induce complete degradation of PD-L1. However, since PD-L1 monoclonal antibody (aPD-L1 antibody) failed to enter the lysosomes and underwent recycling, its effect was very insignificant.

### Experimental Example 5. Analysis of Efficacy of the Peptide-Liposome Complex on T Cell Activity

An evaluation was made as to whether the peptide-liposome complex (10-PD-L1 Lipo) prepared in Example 2 could block and degrade PD-L1 on the cell surface to enhance the ability of T cells to recognize cancer cells. First, CT26 cancer cells were plated in a 6-well cell culture plate at a density of 2 × 10⁵ cells/well. After stabilization for 24 h, cells were treated with PD-L1 monoclonal antibody (aPD-L1 antibody) and the peptide-liposome complex prepared in Example 2 (each 0.5 mg/mL) and cultured at 37° C. for 24 h. Each well was treated with T cells, followed by co-culture for 24 h. The morphology of the CT26 cancer cells in each well was observed under a microscope. The tumor lysis (%) of the CT26 cancer cells and the amount of IFN-γ released (pg/ml) from the T cells were evaluated by ELISA. Untreated CT26 cancer cells were used as a control (“Non-treated”).

For statistical analysis of the experimental data, significant differences in mean values between groups were determined using one-way ANOVA test. * indicates a significant difference at p < 0.05, ** indicates a significant difference at p < 0.01, *** indicates a significant difference atp < 0.001, and N.S indicates no significant difference. Error bars indicate S.D.

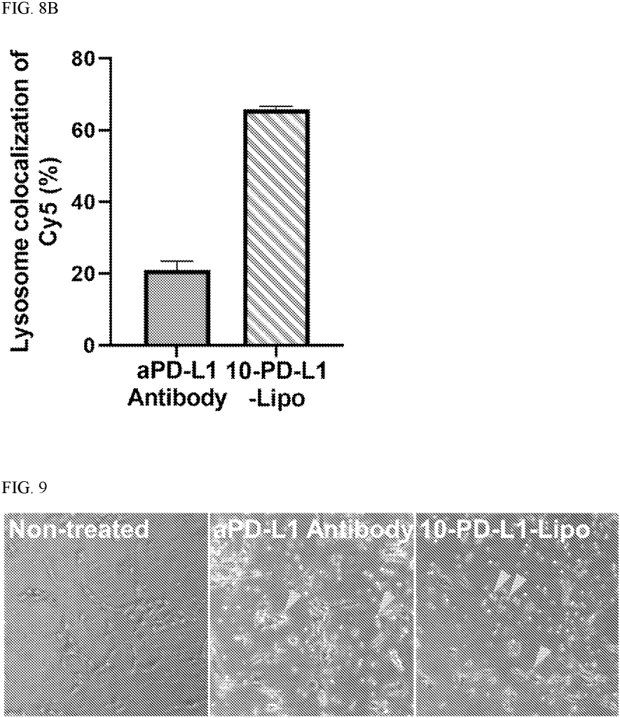

FIG. 9 shows microscopy images of the trans-CT26 cells treated with the peptide-liposome complex (10-PD-L1 Lipo) prepared in Example 2 and co-cultured with T cells and the trans-CT26 cells treated with PD-L1 monoclonal antibody (aPD-L1 antibody) and co-cultured with T cells.

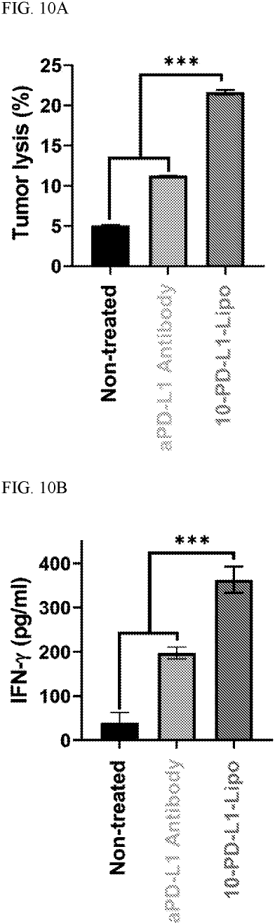

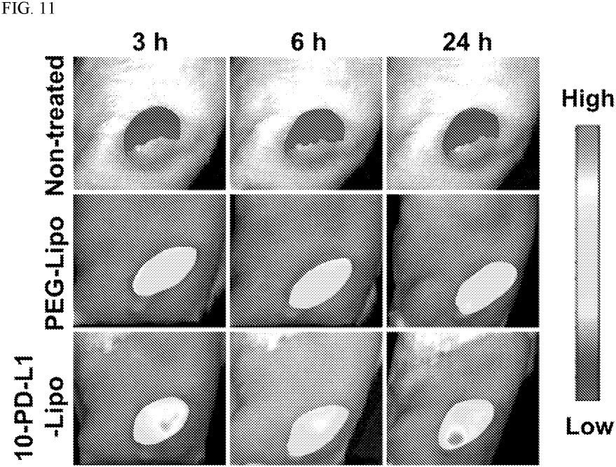

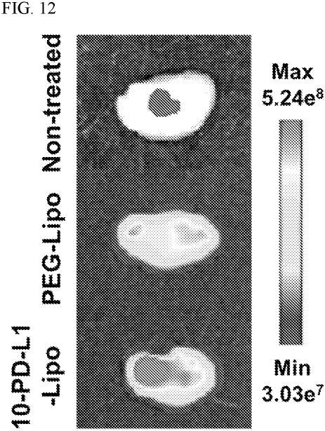

FIG. 10A shows proportions of dead cancer cells and FIG. 10B shows results of quantitative analysis of the concentrations of released interferon gamma (IFN-γ) after co-culture of the trans-CT26 cells treated with the peptide-liposome complex (10-PD-L1 Lipo) prepared in Example 2 with T cells and after co-culture of the trans-CT26 cells treated with PD-L1 monoclonal antibody (aPD-L1 antibody) with T cells.

As shown in FIG. 9, the amount of T cells located around the cancer cells was significantly increased in the group treated with the peptide-liposome complex (10-PD-L1 Lipo) prepared in Example 2 compared to in the other two groups.

As shown in FIGS. 10A and 10B, the tumor lysis (%) of the cancer cells was significantly increased in the group treated with the peptide-liposome complex (10-PD-L1 Lipo) prepared in Example 2 compared to in the other two groups.

In addition, the amount of IFN-y released from T cells was significantly increased in the group treated with the peptide-liposome complex (10-PD-L1 Lipo) prepared in Example 2 compared to in the other two groups.

The above results demonstrated that the peptide-liposome complex (10-PD-L1 Lipo) prepared in Example 2 was bound to and degraded PD-L1 on the surface of cancer cells, prevented the recycling of PD-L1, and significantly increased the ability of T cells to recognize cancer cells compared to aPD-L1 antibody.

### Experimental Example 6. Evaluation of in Vivo Behavior of the Peptide-Liposome Complex

All animal experiments were conducted in accordance with the guidelines of the Korea Institute of Science and Technology (KIST) and were approved by the Institutional Committees. BALB/c mice (5.5 weeks old, 20-25 g, male) purchased from Nara Bio INC (Gyeonggi-do, Korea) were used as animal models. 1 × 10⁶ CT26 cells were inoculated into the left thigh of each of the mice (n=6) to construct establish a cancer animal model. Experiments were conducted when cancer volumes were 250-300 mm³ 5 weeks after inoculation.

When cancer volumes in the cancer animal models reached 250-300 mm³, the peptide-liposome complex prepared in Example 2 (10-PD-L1 Lipo) or the liposomes of Comparative Example 1 were injected into each animal model via the tail vein. After administration, noninvasive near-infrared fluorescence (NIRF) imaging data were obtained using an in vivo fluorescence imaging system (IVIS Luminar III) to assess the in vivo tumor accumulation of the liposomes. 24 h after administration, tumor tissues were excised from the animal models in each group and fluorescence imaging was performed in the same manner as described above.

The tumor tissues obtained from the animal models in each group 24 h after administration were stained with anti-PD-L1 antibody and analyzed by fluorescence microscopy to evaluate whether the peptide-liposome complex (10-PD-L1 Lipo) prepared in Example 2 inhibited the in vivo expression of PD-L1 on the surface of cancer cells.

### Three Groups of Animal Models

Group 1 (Non-treated): Control group without drug administration

Group 2 (PEG-Lipo): Cancer animal models administered 15 mg/kg of the liposomes (PEG-Lipo) of Comparative Example 1 via the tail vein

Group 3 (Experimental group): Cancer animal models administered 15 mg/kg of the peptide-liposome complex (10-PD-L1 Lipo) prepared in Example 2 via the tail vein

FIG. 11 shows the results of in vivo fluorescence analysis for the colorectal cancer animal models in Group 3 administered the peptide-liposome complex (10-PD-L1 Lipo) prepared in Example 2, Group 2 administered the liposomes (PEG-Lipo) of Comparative Example 1, and non-treated Group 1.

As shown in FIG. 11, the amount of the peptide-liposome complex (10-PD-L1 Lipo) prepared in Example 2 accumulated in the tumor was significantly large in Group 3 compared to those in Groups 1 and 2.

FIG. 12 shows the results of fluorescence analysis for the tumor tissues excised from the animal models in Groups 1, 2 and 3. As shown in FIG. 12, a significantly large amount of the drug (10-PD-L1 Lipo) was accumulated in the tumor tissue from Group 3 administered the peptide-liposome complex prepared in Example 2 (10-PD-L1 Lipo).

That is, the peptide-liposome complex (10-PD-L1 Lipo) prepared in Example 2 was not cleared in vivo through a combination of the enhanced permeability and retention (EPR) effect and the PD-L1 binding effect and could be accumulated in a significantly large amount in tumor compared to the conventional drug delivery vectors.

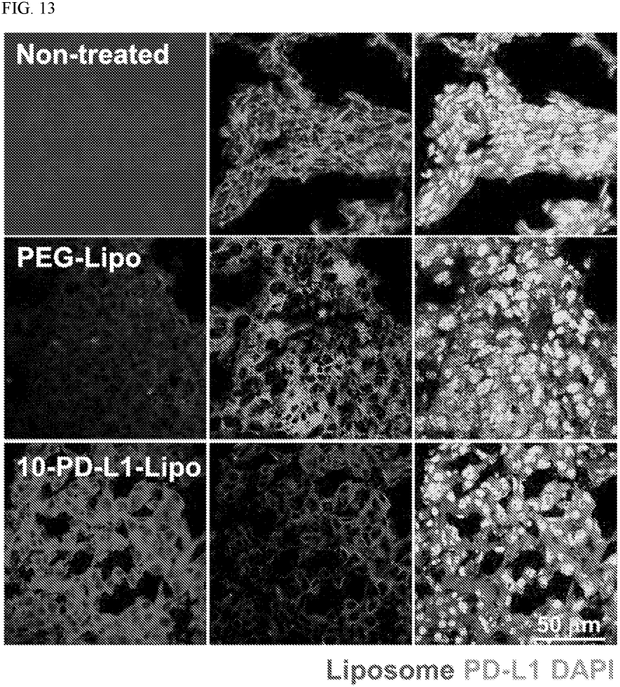

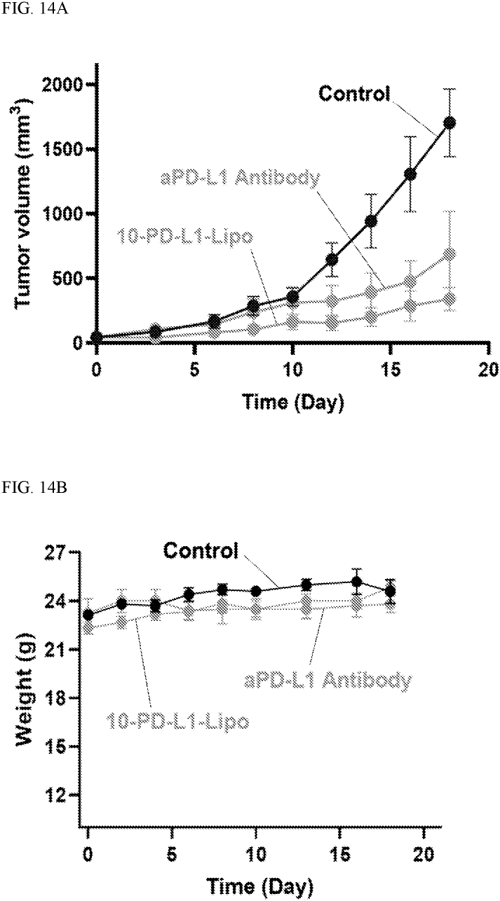

FIG. 13 shows fluorescence microscopy images of the tumor tissues excised from the animal models in Groups 1, 2 and 3 after staining with anti-PD-L1 antibody.

As shown in FIG. 13, the expression level of PD-L1 (green) in the tumor tissue of the animal model in Group 3 treated with the peptide-liposome complex (10-PD-L1 Lipo) prepared in Example 2 was significantly decreased compared to those in the animal models in Groups 1 and 2. Therefore, the peptide-liposome complex (10-PD-L1 Lipo) prepared in Example 2 has a significantly superior therapeutic effect in vivo on cancer compared to conventional liposomes.

### Experimental Example 7. Evaluation of Anticancer Effect and Toxicity of the Peptide-Liposome Complex

The anticancer effect of the peptide-liposome complex (10-PD-L1 Lipo) prepared in Example 2 was analyzed and an investigation was made as to whether the peptide-liposome complex caused side effects during the treatment period. Specifically, all animal experiments were conducted in accordance with the guidelines of the Korea Institute of Science and Technology (KIST) and were approved by the Institutional Committees. BALB/c mice (5.5 weeks old, 20-25 g, male) purchased from Nara Bio INC (Gyeonggi-do, Korea) were used as animal models. 1 × 10⁶ CT26 cells were inoculated into the left thigh of each of the mice (n=6) to construct establish a cancer animal model. Experiments were conducted when cancer volumes were 50-70 mm³ 5 weeks after inoculation.

When cancer volumes in the cancer animal models reached 50-70 mm³, the peptide-liposome complex prepared in Example 2 (10-PD-L1 Lipo) or the liposomes of Comparative Example 1 were injected into each animal model via the tail vein every 3 days. From immediately after injection (day 0), changes in body weight and tumor tissue volume of each group were measured every 2 days and survivals were analyzed. The tumor tissue volume (V; mm³) was calculated as 0.53 × largest diameter × (smallest diameter)².

20 days after administration, tumor tissues were excised from the animal models in each group and apoptoses in the tumor tissues were analyzed by TUNEL staining.

The proportions of T cells and regulatory T cells in the tumor tissues obtained 20 days after administration were analyzed. Specifically, the proportions of T cells and regulatory T cells in the tumor tissues excised from each group were analyzed by flow cytometry. For the analysis of T cells and regulatory T cells, monocytes were isolated from the tumor tissues using a tumor dissociation kit (Miltenyi Biotec) according to the manufacturer’s protocol. Next, the monocytes were cultured with Fc block for 5 min to avoid non-specific binding and stained with CD45, CD3, and CD8 antibodies as T cell markers for analysis. For regulatory T cells, CD3, CD4, and FoxP3 antibodies were used for staining

### Three Groups of Animal Models

Group 1 (Non-treated): Control group without drug administration

Group 2 (PEG-Lipo): Cancer animal models administered 15 mg/kg of the liposomes (PEG-Lipo) of Comparative Example 1 via the tail vein

Group 3 (Control prodrug): Cancer animal models administered 15 mg/kg of the peptide-liposome complex (10-PD-L1 Lipo) prepared in Example 2 via the tail vein

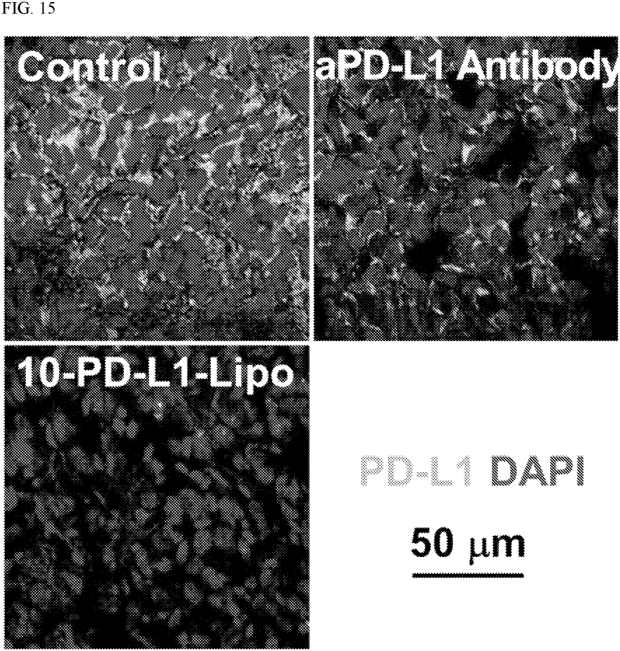

FIG. 14A shows changes in tumor volume (V; mm³) in the animal models in Groups 1, 2, and 3 during the treatment period and FIG. 14B shows changes in the weight of the animal models in Groups 1, 2, and 3 during the treatment period.

As shown in FIGS. 14A and 14B, 18 days after drug administration, the tumor volumes in the animal models in Group 3 (10-PD-L1 Lipo), Group 1, and Group 2 were 339.86 ± 90.17 mm³, 1703.88 ± 262.35 mm³, and 687.25 ± 329.12 mm³, respectively. These results indicate that tumor growth was more significantly inhibited in Group 3 administered the peptide-liposome complex (10-PD-L1 Lipo) prepared in Example 2 than in the other groups. No significant change in body weight was observed in Group 3 administered the peptide-liposome complex (10-PD-L1 Lipo) prepared in Example 2 during the treatment period, indicating that the peptide-liposome complex is a stable material with no side effects.

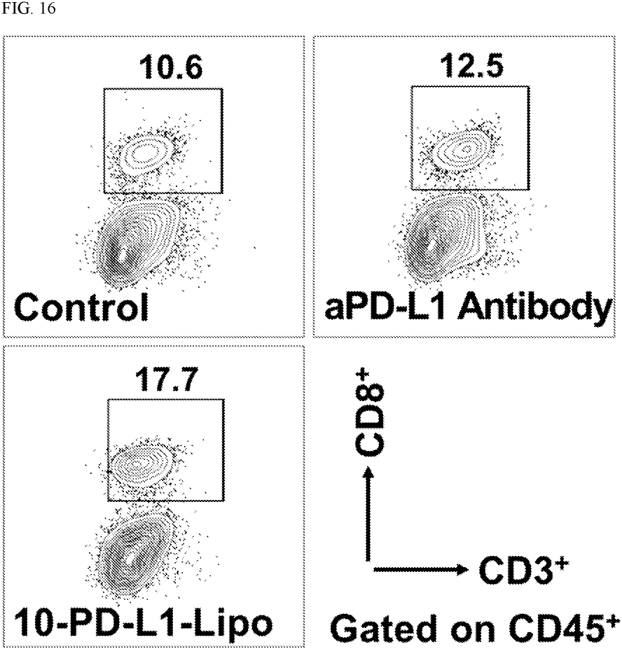

FIG. 15 shows TUNEL-stained tumor tissues excised from the animal models in Groups 1, 2, and 3 20 days after drug administration.

As shown in FIG. 15, apoptoses in the tumor tissues were evaluated by TUNEL staining. As a result, a significantly high level of apoptosis of tumor cells occurred in Group 3 treated with the peptide-liposome complex (10-PD-L1 Lipo) prepared in Example 2, indicating that the peptide-liposome complex (10-PD-L1 Lipo) prepared in Example 2 exhibits a significantly superior anticancer effect compared to the conventional anticancer drug.

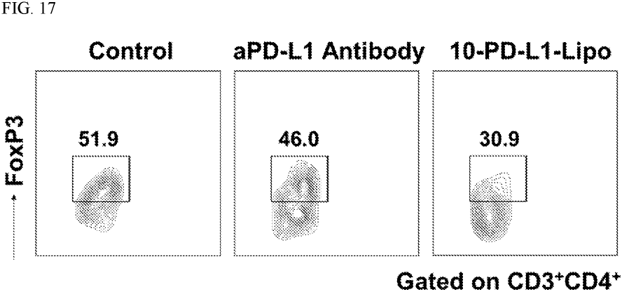

FIG. 16 shows the proportions of T cells expressing CD45, CD3, and CD8 in the tumor tissues excised from the animal models in Groups 1, 2, and 3 20 days after drug administration, which were analyzed by flow cytometry. FIG. 17 shows the proportions of regulatory T cells expressing CD3, CD4, and FoxP3 in the tumor tissues excised from the animal models in Groups 1, 2, and 3 20 days after drug administration, which were analyzed by flow cytometry.

As shown in FIG. 16, the proportion of T cells in Group 3 treated with the peptide-liposome complex (10-PD-L1 Lipo) prepared in Example 2 was 17.7%, which was significantly higher than those in Group 1 (10.6%) and Group 2 (12.5%).

As shown in FIG. 17, the proportion of regulatory T cells in Group 3 treated with the peptide-liposome complex (10-PD-L1 Lipo) prepared in Example 2 was 30.9%, which was significantly lower than those in Group 1 (51.9%) and Group 2 (46%).

In conclusion, when administered in vivo, the peptide-liposome complex (10-PD-L1 Lipo) prepared in Example 2 binds to and degrades PD-L1 on the surface of cancer cells to significantly increase the ability of T cells to recognize cancer cells, and as a result, adaptive immunity is significantly activated, resulting in an increase in the proportion of T cells infiltrating into tumor tissues and a reduction in the proportion of immunosuppressive regulatory T cells in tumor tissues.

What is claimed is:

1. A peptide-liposome complex composed of a lipid bilayer comprising (a) a first phospholipid, (b) a second phospholipid containing PEG, (c) cholesterol, and (d) a lipid conjugate consisting of the second phospholipid and a peptide having the amino acid sequence set forth in SEQ ID NO: 1.

2. The peptide-liposome complex according to claim 1, wherein the first phospholipid is selected from the group consisting of phosphatidylcholine (PC), phosphatidic acid (PA), phosphatidylethanolamine (PE), phosphatidylglycerol (PG), phosphatidylserine (PS), phosphatidylinositol (PI), dimyristoylphosphatidylcholine (DMPC), distearoylphosphatidylcholine (DSPC), dioleoylphosphatidylcholine (DOPC), dipalmitoylphosphatidylcholine (DPPC), dimyristoylphosphatidylglycerol (DMPG), distearoylphosphatidylglycerol (DSPG), dioleoylphosphatidylglycerol (DOPG), dipalmitoylphosphatidylglycerol (DPPG), dimyristoylphosphatidylserine (DMPS), distearoylphosphatidylserine (DSPS), dioleoylphosphatidylserine (DOPS), dipalmitoylphosphatidylserine (DPPS), dioleoylphosphatidylethanolamine (DOPE), palmitoyloleoylphosphatidylcholine (POPC), palmitoyloleoylphosphatidylethanolamine (POPE), dioleoylphosphatidylethanolamine 4-(N-maleimidomethyl)-cyclohexane-1-carboxylate (DOPE-mal), dipalmitoylphosphatidylethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidylethanolamine (SOPE), 1,2-dielaidoyl-sn-glycero-3-phosphoethanolamine (transDOPE), cardiolipin, and mixtures thereof.

3. The peptide-liposome complex according to claim 1, wherein the second phospholipid is 1,2-distearoyl-sn-glycero-3-phosphoethanolamine-N-[methoxy(polyethylene glycol)2000] (DSPE-mPEG2000) or 1,2-disteroyl-sn-glycero-3-phosphoethanolamine-N-[maleimide(polyethylene glycol)2000] (DSPE-PEG2000-MAL).

4. The peptide-liposome complex according to claim 1, wherein the lipid conjugate consisting of the second phospholipid and a peptide having the amino acid sequence set forth in SEQ ID NO: 1 is present in an amount of 5 to 30 mol%, based on the total moles of all lipids in the peptide-liposome complex.

5. The peptide-liposome complex according to claim 1, wherein the peptide-liposome complex is a spherical hollow body having an average diameter of 50 to 300 nm and composed of a lipid bilayer membrane.

6. The peptide-liposome complex according to claim 1, further comprising an anticancer agent.

7. A composition for diagnosing cancer comprising the peptide-liposome complex according to claim 1 and a fluorescent molecule.

8. The composition according to claim 7, wherein the cancer is derived from cancer cells overexpressing PD-L1 on the cell surface.

9. A pharmaceutical composition for preventing or treating cancer comprising the peptide-liposome complex according to claim 1.

10. The pharmaceutical composition according to claim 9, wherein the cancer is selected from the group consisting of gastric cancer, lung cancer, non-small cell lung cancer, breast cancer, ovarian cancer, liver cancer, bronchial cancer, nasopharyngeal cancer, laryngeal cancer, pancreatic cancer, bladder cancer, colon cancer, cervical cancer, bone cancer, non-small cell bone cancer, hematologic malignancy, skin cancer, head or neck cancer, uterine cancer, rectal cancer, perianal cancer, fallopian tube cancer, endometrial cancer, vaginal cancer, vulvar cancer, Hodgkin’s disease, esophageal cancer, small intestine cancer, endocrine gland cancer, thyroid cancer, parathyroid cancer, adrenal cancer, soft tissue sarcoma, urethral cancer, penile cancer, prostate cancer, chronic or acute leukemia, lymphocytic lymphoma, kidney or ureter cancer, renal cell carcinoma, renal pelvic carcinoma, salivary gland cancer, sarcoma, pseudomyxoma, hepatoblastoma, testicular cancer, glioblastoma, lip cancer, ovarian germ cell tumor, basal cell carcinoma, multiple myeloma, gallbladder cancer, choroidal melanoma, ampulla of Vater cancer, peritoneal cancer, tongue cancer, small cell cancer, pediatric lymphoma, neuroblastoma, duodenal cancer, ureteral cancer, astrocytoma, meningioma, renal pelvis cancer, vulvar cancer, thymus cancer, central nervous system (CNS) tumor, primary central nervous system lymphoma, spinal cord tumor, brainstem glioma, pituitary adenoma, and combinations thereof.
